(19) United States
(12) Patent Application Publication
(10) Pub. No.: US 2004/0138587 A1
(43) Pub. Date: Jul. 15, 2004

(54) Specimen collection instrument with inflatable bag

(76) Inventor: William Lawrence Lyons IV, Wallingford, CT (US)

Correspondence Address: GREENBERG TRAURIG, LLP, 885 3RD AVENUE, NEW YORK, NY, 10022, US

(21) Appl. No.: 10/621,274

(22) Filed: Jul. 17, 2003

Related U.S. Application Data
(60) Provisional application No. 60/440,170, filed on Jan. 15, 2003.

(51) Int. Cl. A61B 10/00

(52) U.S. Cl. 600/562

(57) ABSTRACT

The present invention relates, in general, to surgical instruments for collecting and/or removing tissue (or other specimens taken from a body) and, more particularly, to endoscopic and/or laparoscopic surgical instruments such as pouches or specimen retrieval bags for the removal of tissue or other specimens, as indicated above, through a small incision.

### CROSS-REFERENCE TO RELATED APPLICATIONS

This application claims the benefit under 35 U.S.C. 119(e) of U.S. Provisional Application Serial No. 60/440,170, filed Jan. 15, 2003

### FIELD OF THE INVENTION

The present invention relates, in general, to surgical instruments for collecting and/or removing tissue (or other specimens taken from a body) and, more particularly, to endoscopic and/or laparoscopic surgical instruments such as pouches or specimen retrieval bags for the removal of tissue or other specimens, as indicated above, through a small incision.

For the purposes of the present application the term “gas” is intended to refer to any desired element, molecule, composition, or mixture in a gaseous state (e.g., “air”).

Further, for the purposes of the present application the term “gas-tight” is intended to refer to “sealable from ambient pressure”.

Further still, for the purposes of the present application the term “valve” is intended to refer to a mechanism for selectively holding and releasing gas pressure.

### SUMMARY OF THE INVENTION

The present invention is directed to a surgical instrument for collecting and/or removing a tissue (or other specimen) from a patient. One embodiment of the present invention relates to a surgical instrument which comprises an inner tube housed within an outer tube, with the inner tube being movable through the outer tube. The inner tube includes a passageway through which air can flow. The inner tube, at one end, is in fluid communication with a sample collection bag, which, prior to deployment, resides within the outer tube. The inner tube, when moved through the outer tube, pushes the sample collection bag out of the outer tube. At the other end, the inner tube is fitted within, or joined to, a handle. Components to pump air through the inner tube and into the ducts and veins of the bag are provided on the handle or near the handle. Such components may include bulbs, which when compressed, displace air out of the bulb and through the tube. A canister containing pressurized air may also he employed. Other arrangements are provided in the “Detailed Description” section of this disclosure.

In one embodiment, a cinch string extends through the inner tube, from a sealing plug that seals an opening in the inner tube and/or handle, to the bag. At the bag, the cinch string extends around the periphery of the top of the bag, inside of the duct, tied in a noose or noose-like knot. In another embodiment, a washer like ring may be used as opposed to running the string through a knot. The string may instead be permanently affixed to the bag or bag seal area, which would eliminate the need for any type of knot. After the sample has been collected, the sealing plug can be pulled, which will tighten the cinch string and close the bag.

In one embodiment the sample collection bag is provided with a wall made of a flexible material. The bag is closed at one end and open at another end, through which a sample is deposited into the bag. The bag may be further provided with at least one duct, positioned between the inner and outer sidewall of the bag, which provides a passageway through which air can flow and inflate the duct. In one specific embodiment, the bag is provided with a network of ducts. In yet a more specific embodiment, a duct extends around the periphery of the bag and a number of veins, in fluid communication with the duct, extend longitudinally. The duct is in fluid communication with the inner tube, which as indicated above, is itself in fluid communication with an inflation device, which when activated, causes air to flow through the tube, into the duct(s), to inflate the bag, which causes the bag to open, and unfurl the bag. With the bag opened, the surgeon, or other medical personnel, can place a tissue specimen into the sample collection bag. After the sample has been collected, the bag can be deflated by removing the sealing plug, or, as provided in one specific embodiment, by operating a deflation pump or other methods described herein. Removal of the plug tugs on the cinch string, which nooses the bag closed.

The inner tube can be removed from the bag after specimen collection and cinching. This allows the surgeon to leave the bag inside of the cavity, continue using the port with other instruments, or remove the bag at the end of the procedure. To remove the tube from the bag the inner tube may be pulled up through the outer tube until the cinched bag comes into contact with the outer tube. The outer tube may act as a stop, therefore causing the inner tube separate from the bag stem. The inner and outer tubes may be removed and the string may be pulled into the cavity with graspers (or a similar mechanism) from a different port.

### BRIEF DESCRIPTION OF THE DRAWINGS

FIG. 1 is a perspective view showing one embodiment of the present invention;

FIG. 2 is a perspective view showing a sample collection bag employed with an embodiment of the present invention;

FIG. 3 is a perspective view showing another embodiment of the present invention;

FIG. 4 is a perspective view showing another embodiment of the present invention;

FIG. 5 is a perspective view showing another embodiment of the present invention;

FIG. 6 is a perspective view showing another embodiment of the present invention;

FIG. 7 is a cross sectional view showing the sample collection bag of an embodiment of the present invention in a pre-deployed state.

FIG. 8 is a side cross sectional view showing another embodiment of a sample collection bag employed in the present invention;

FIG. 9 is a side cross sectional view showing yet another embodiment of a sample collection bag employed in the present invention;

FIG. 10 is a perspective view showing another embodiment of the present invention that includes a knife blade; and

FIG. 11 is a side elevational view of the FIG. 10 embodiment.

Among those benefits and improvements that have been disclosed, other objects and advantages of this invention will become apparent from the following description taken in conjunction with the accompanying figures. The figures constitute a part of this specification and include illustrative embodiments of the present invention and illustrates various objects and features thereof.

### DETAILED DESCRIPTION OF THE INVENTION

Detailed embodiments of the present invention are disclosed herein; however, it is to be understood that the disclosed embodiments are merely illustrative of the invention that may be embodied in various forms. In addition, each of the examples given in connection with the various embodiments of the invention are intended to be illustrative, and not restrictive. Further, the figures are not necessarily to scale, some features may be exaggerated to show details of particular components. Therefore, specific structural and functional details disclosed herein are not to be interpreted as limiting, but merely as a representative basis for teaching one skilled in the art to variously employ the present invention.

In one embodiment a specimen collection apparatus is provided, comprising: a collapsible specimen retrieval bag having an open end which can be cinched shut to contain a specimen therein; a housing for holding the specimen retrieval bag in a collapsed state; a mechanism for deploying the specimen retrieval bag from the housing; and a mechanism for shaping the open end of the specimen retrieval bag, after the specimen retrieval bag is deployed from the housing, to form an aperture large enough to receive the specimen into the specimen retrieval bag; wherein the mechanism for shaping the open end of the specimen retrieval bag includes at least one substantially gas-tight duct associated with the specimen retrieval bag, which duct is disposed at a position selected from the group of: (a) on an inside surface of the specimen retrieval bag; (b) on an outside surface of the specimen retrieval bag; and (c) between the inside surface of the specimen retrieval bag and the outside surface of the specimen retrieval bag.

In one example the duct may be disposed adjacent the open end of the specimen retrieval bag.

In another example the duct may form a generally circular ring when gas pressure in the duct is greater than gas pressure outside the duct.

In another example the apparatus may further comprise a mechanism for supplying gas to the duct in order to inflate the duct.

In another example the mechanism for supplying the gas to the duct may be adapted to provide incremental inflation of the duct.

In another example the mechanism for supplying the gas to the duct may be selected from the group including, but not limited to: (a) an electrical pump; (b) a hand-operated pump; (c) a container of compressed gas; and (d) a source within an operating room.

In another example the hand operated pump may be selected from the group including, but not limited to: (a) a bulb-type pump; and (b) a syringe.

In another example the apparatus may further comprise a mechanism for permitting gas pressure in the duct to be reduced in order to deflate the duct.

In another example the mechanism for permitting gas pressure in the duct to be reduced may be adapted to provide incremental deflation of the duct.

In another example the mechanism for permitting gas pressure in the duct to be reduced may comprise a valve.

In another example the apparatus may further comprise a mechanism for deflating the duct by decreasing gas pressure therein.

In another example the mechanism for deflating the duct may comprise a mechanism for pumping gas out of the duct.

In another example the mechanism for pumping gas out of the duct may be adapted to provide incremental deflation of the duct.

In another example the mechanism for pumping gas out of the duct may be selected from the group including, but not limited to: (a) an electrical pump; (b) a hand-operated pump; and (c) a source within an operating room.

In another example the hand-operated pump may be selected from the group including, but not limited to: (a) a bulb-type pump; and (b) a syringe.

In another example the gas may be air.

In another example the apparatus may further comprise a mechanism for cinching shut the open end of the specimen retrieval bag.

In another example the mechanism for cinching shut the open end of the specimen retrieval bag may include a string running at least partially through the duct, which string may be pulled to cinch shut the open end of the specimen retrieval bag.

In another example the housing may comprise a first elongated tube with two ends.

In another example the mechanism for deploying the specimen retrieval bag may comprise a second elongated tube with an outside diameter smaller than an inside diameter of the first elongated tube, wherein the second elongated tube is configured to push the specimen retrieval bag out of one of the ends of the first elongated tube.

In another example the apparatus may comprise a knife disposed on the housing.

In another example the knife may be disposed on an end of the housing opposite that from which the specimen retrieval bag is deployed.

In another embodiment a specimen collection apparatus is provided, comprising: a collapsible specimen retrieval bag having an open end which can be cinched shut to contain a specimen therein; a housing for holding the specimen retrieval bag in a collapsed state, which housing comprises a first elongated tube with two ends; a mechanism for deploying the specimen retrieval bag from the housing, which mechanism for deploying the specimen retrieval bag from the housing comprises a second elongated tube with an outside diameter smaller than an inside diameter of the first elongated tube, wherein the second elongated tube is configured to push the specimen retrieval bag out of one of the ends of the first elongated tube; a substantially gas-tight duct associated with the specimen retrieval bag for shaping the open end of the specimen retrieval bag, after the specimen retrieval bag is deployed from the housing, to form an aperture large enough to receive the specimen into the specimen retrieval bag; a mechanism for inflating the duct by supplying gas to the duct; a mechanism for deflating the duct by permitting gas pressure in the duct to be reduced; and a mechanism for cinching shut the open end of the specimen retrieval bag; wherein the duct is disposed at a position selected from the group of: (a) on an inside surface of the specimen retrieval bag; (b) an outside surface of the specimen retrieval bag; and (c) between the inside surface of the specimen retrieval bag and the outside surface of the specimen retrieval bag; wherein the duct is disposed adjacent the open end of the specimen retrieval bag; and wherein the duct forms a generally circular ring when gas pressure in the duct is greater than gas pressure outside the duct.

In one example the mechanism for cinching shut the open end of the specimen retrieval bag may include a string running at least partially through the duct, which string may be pulled to cinch shut the open end of the specimen retrieval bag.

In another example: (a) the string may run through the first elongated tube and the second elongated tube; and (b) gas may be supplied to the duct through the first elongated tube and the second elongated tube.

In another example the apparatus may further comprise a mechanism for pumping gas out of the duct.

In another example gas may be pumped out of the duct through the first elongated tube and the second elongated tube.

In another example the apparatus may further comprise a stem in fluid communication with the duct, wherein gas passes through the stem during inflation and deflation.

In another example the string may run through the stem.

In another example the stem may be attached to the specimen retrieval bag at a reinforced portion of the specimen retrieval bag.

In another example the mechanism for deflating the duct by permitting gas pressure in the duct to be reduced may comprise a valve having a gas blocking element seatable in an interference fit with a seating surface.

In another example the valve may be operatively connected to the string such that the valve permits gas pressure in the duct to be reduced after the string is pulled.

In another example the apparatus may further comprise a backflow prevention mechanism operatively connected to the string for substantially prohibiting a flow of gas back into the duct after the string is pulled past a predetermined position during the cinching shut of the open end of the specimen retrieval bag.

In another example the backflow prevention mechanism may comprise a valve having a gas blocking element seatable in an interference fit with a seating surface.

In another example the apparatus may further comprise a mechanism for unfurling the specimen retrieval bag, after the specimen retrieval bag is deployed from the housing.

In another example the mechanism for unfurling the specimen retrieval bag may comprise at least one vein extending from the duct, which vein is in fluid communication with the duct.

In another example the vein may extend generally away from the open end of the specimen retrieval bag and towards a closed end of the specimen retrieval bag.

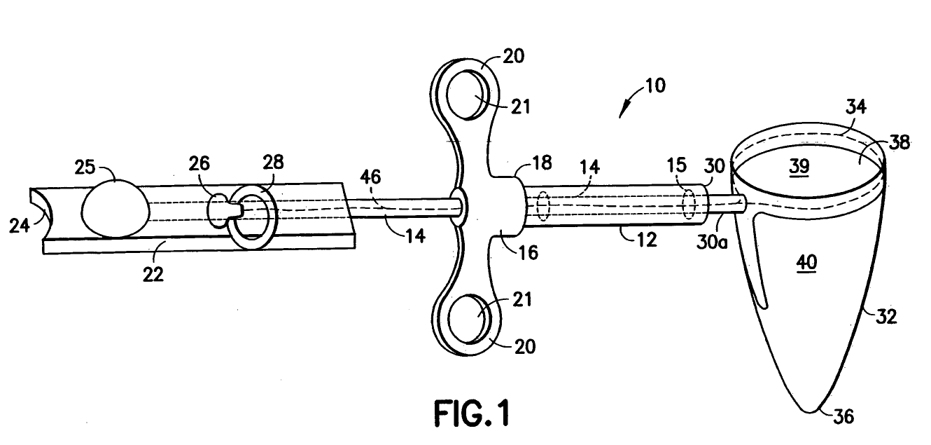

FIG. 1 shows a specimen collection instrument 10 in a deployed state, that is, where collection bag 32 has been moved out of the outer tube 12, and had been inflated, in the course of actions that will be described later herein. The specimen collection instrument 10 includes an outer tube 12 housing an inner tube 14. Outer tube 12 is provided with a handle 16 at a proximal end 18 of the outer tube. Handle 16 has pair of lobes 20 with openings 21 through which the operator of the instrument can insert their fingers. Handle 16 is fixedly attached to the outer tube 12. Outer tube 12 defines an open space through its interior, in which inner tube 14 is housed. The outer tube 12 provides a passageway for inner tube 14 to move through. O-rings 15 or other sealing mechanism(s) may be positioned in the outer tube, between the outer tube and the inner tube, in order to create an interference fit between the two (to seal the space between inner tube and outer tube).

As shown in FIG. 1, the inner tube 14 is provided at its proximal end with a handle 22. Handle 22 is hollow in its interior, and receives the inner tube 14. A finger indent 24, on which a thumb or other digit can he positioned, is provided on the handle 22. A bulb 25 is positioned on the handle 22, such as by recessing it into the handle, and the bulb 25 is in fluid communication with the inner tube 14. The bulb 25 acts as a pump in order to inflate the sample collection pouch 32. The bulb 25 is constructed of a soft, deformable material, such as an elastomer. The compression of the bulb displaces air residing in the bulb, forcing air through the inner tube and into the pouch to inflate it. After the compression force is removed (which may be manually applied), the bulb returns to its original shape. The pump has valve (not shown) to provide for incremental inflation and to allow air to be drawn into the bulb.

The handle 22 also is provided with a sealing plug 26 formed of a suitable sealing material. Sealing plug 26 is provided with a pull ring 28 to enable the operator to pull the plug 26. The inside of the sealing plug, which faces the interior of the handle, is provided with a loop or other structure (which is not shown) to which a cinch string 46 is attached. The inner tube 14 extends from the proximal end where handle 22 is located, through the outer tube 12, towards the distal end 30a. The inner tube is hollow. At its distal end, the inner tube is provided with a closed wall (not shown) having an opening 33 to receive a stem from the sample collection bag 32.

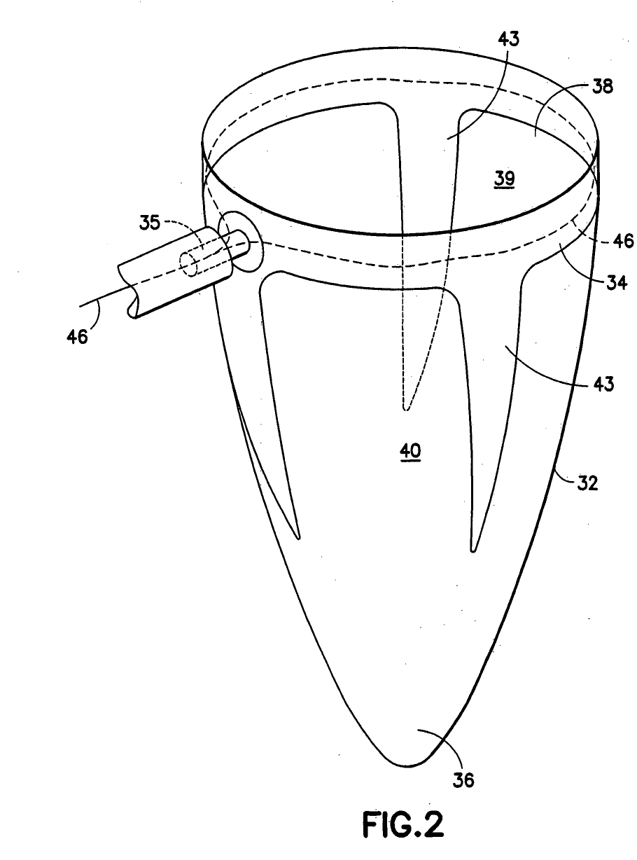

The unactuated sample collection bag 32 resides within the outer tube 12, near the distal end 30 thereof. (See FIG. 2). The stem 35 is in fluid communication with a duct 34 formed between the inner wall and outer wall of the bag (See FIG. 2). The stem 35 extends from the bag and is inserted into opening 33 in the inner tube 14. (In an alternative arrangement, the stem 35 is attached to the inner tube 14, as opposed to being joined to the bag, with which, in this instance, it forms a detachable, yet sealed, connection.) Stem 35 should be formed of a durable, resilient material to support manipulation of the pouch before, during, and after sample collection, through manipulation of the inner tube.

As seen in FIG. 2, the sample collection bag 32 is provided with a closed lower end 36 and an open upper end 38. The collection bag 32 has an inner wall 39 and an outer wall. At least one duct 34 is formed between the inner wall and outer wall. As shown in FIG. 2, the duct 34 extends around the perimeter of the bag 32, with a number of veins 43, in fluid communication with the duct 34, and extending longitudinally from the duct 34. Duct 34 is in communication with the stem 35. Cinch string 46, which as indicated above is attached to the sealing plug 26 located on the handle 22 and which extends through the inner tube 14, through the opening 33 in closed wall (not shown) at the distal end of the inner tube 14, passes into and around the duct, and loops around the cinch string 46 at the junction of the stem 35 and duct 46 forming a loop. The noose, or knot, should be sized so that the knot is not drawn up into the stem during the cinching operation. At the appropriate time, when the plug 26 is pulled, the cinch string 46 is tightened, cinching the sample collection bag 32 closed.

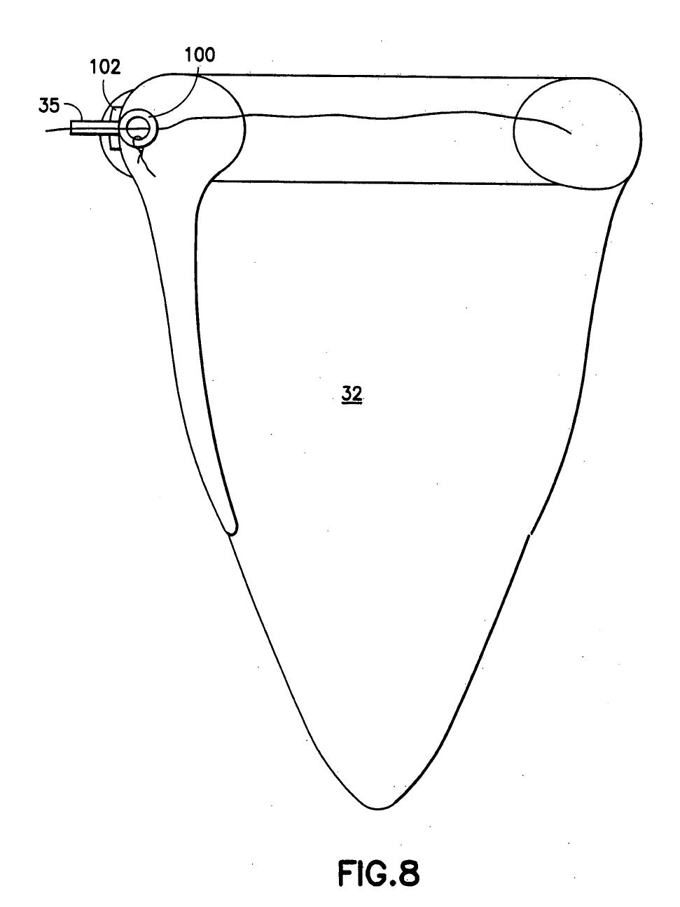

In an alternative embodiment, shown in FIG. 8. a ring or washer 100 is positioned in the duct. Cinch string 46 passes through the opening in the washer 100, around the duct, as described above, and the end of cinch string 46 is then affixed to the washer. With this arrangement, the cinch string will tighten around the periphery of the bag in order to close it. In another embodiment, the washer is affixed to a membrane which seals around the stem. Affixing the washer to the membrane may aid in the cinching operation. In another embodiment, the cinch string 46 may affix to the membrane directly (i.e., without use of a washer).

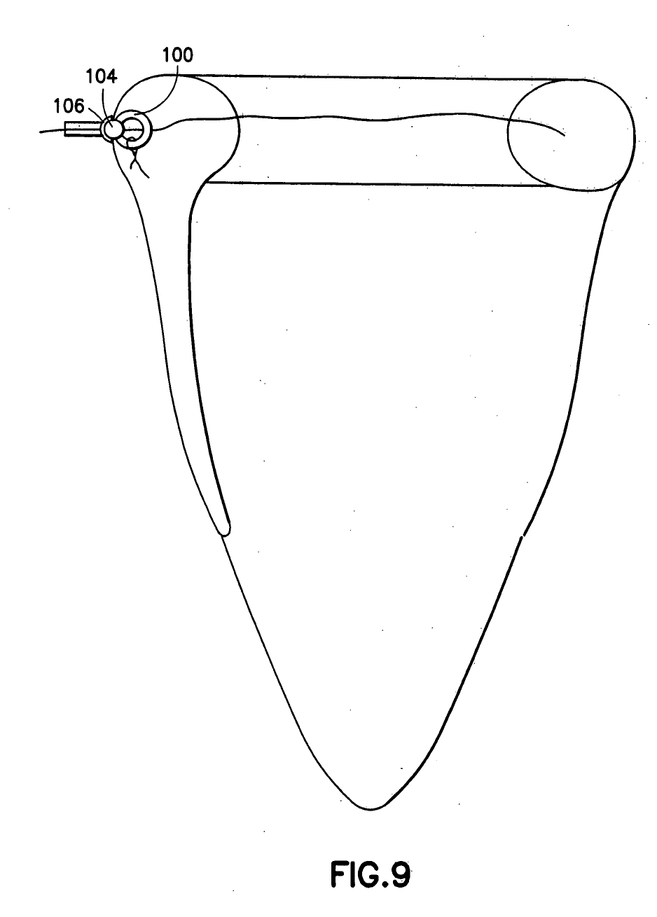

In yet another embodiment, shown in FIG. 9, a ball stop 104 is positioned on the cinch string 46. In yet another embodiment, it is affixed to it. The ball stop is sized greater than the opening in the stem 35. The ball stop is positioned on the cinch string 46 at a location where it will not encounter the stem 35 until the bag is essentially fully closed. The ball stop provides a positive indicator that the bag is fully closed. The stem may be provided with a ball seat 106, in which the ball is positioned when the positive stop has been attained. The seat may be provided with gripping sidewalls, so that when the ball is positioned in the seat, it snaps into place, and blocks air from reentering the duct.

In yet another embodiment, the duct may have a surface profile that aids in the closing of the bag. Such profiles include a switchback pattern, such as found on an accordion, variations in surface thickness, and/or cross hatching, to aid in cinching of the bag.

In operation, the surgical device is introduced into a body cavity, (e.g., through a port or opening which has been opened by the surgeon) in a state in which the bag has not been actuated. The pouch is deployed by pushing handle 22 to slide the inner tube 14 through the outer tube 12, causing the bag to be pushed out of the outer tube 12. After deployment, the bulb 25 on the handle 22 is compressed, forcing air through the inner tube 14, stem 35, and into duct 34 (and veins 43, if provided). This inflates the bag, causing it to open and unfurl substantially completely, a state in which it is sufficiently open to receive a sample of tissue or other specimen.

The tissue or other specimen is then placed in the pouch. The air is then allowed to escape from the ducts (and veins) in the bag by removing the sealing plug 26. Pulling on the sealing plug 26 after its removal also tugs at the cinch string 46, cinching the top of the bag closed. The cinch string 46 may now be pulled, closing the mouth of the pouch. The cinch string may be cut. The inner tube can be removed from the bag after specimen collection and cinching. This allows the surgeon to leave the bag inside of the cavity, continue using the port with other instruments, or remove the bag at the end of the procedure. To remove the tube from the bag the inner tube may be pulled up through the outer tube until the stem contacts the outer tube (the outer tube acts as a stop, therefore causing the inner tube to separate from the bag stem). The inner and outer tubes may be removed and the string may be pulled into the cavity with graspers (or a similar mechanism) from a different port. The device may be removed from the port. The port may be removed from the cavity. The bag may then be removed through the incision. The incision may need to be opened further to accommodate larger specimens.

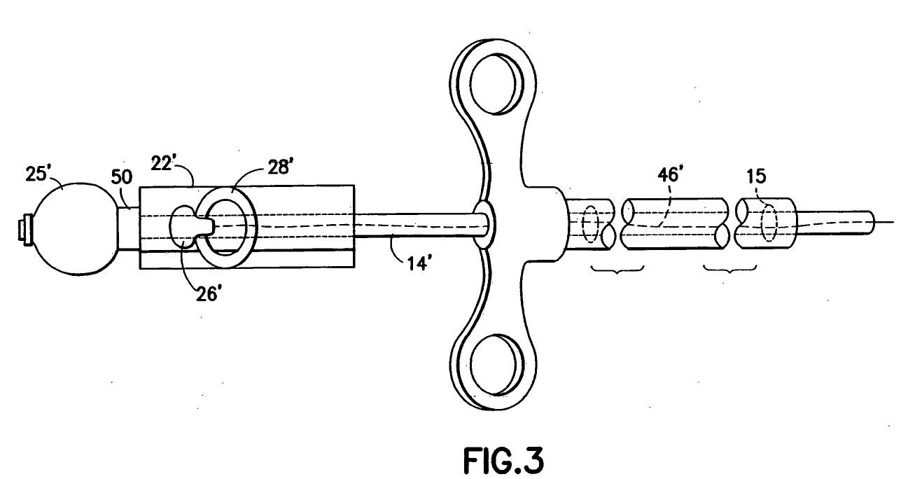

FIG. 3 shows an alternative embodiment. In this embodiment, handle 22′ is positioned over the inner tube 14′, as it is in FIG. 1. A bulb 25′, positioned on the handle 22′, is in fluid communication with the inner tube 14′ and is joined thereto by fitting 50. Bulb 25′ can be formed of a soft compressible elastomeric material, as described above. Cinch string 46′ extends from the bag, in the manner described above, through the inner tube, the fitting 50, and the handle, and terminates in sealing plug 26′ positioned in the handle.

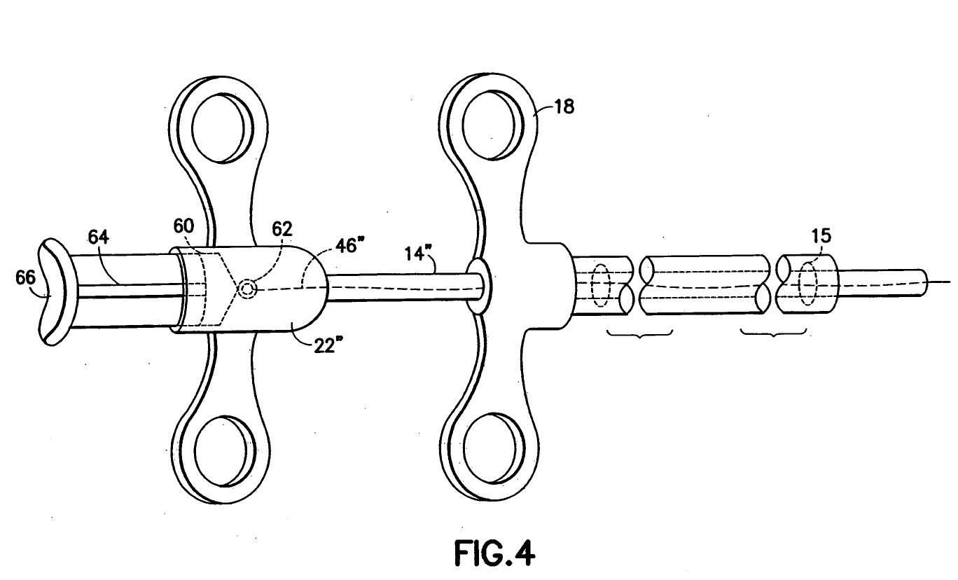

FIG. 4 depicts yet another embodiment of the present invention where handle 22″ is a hollow body in fluid communication with the inner tube 14″. As shown in the figure, the cross sectional area of the hollow body portion body may be greater than the cross sectional area of the inner tube and may hold a volume of gas equal or greater than the volume of the ducts and veins of the bag 32. The handle is open at its proximal end and is provided with an air-tight seal by plunger 60. On the interior side of the plunger 60, a loop 62 is provided to which the cinch string 46″ is attached. A stem 64 extends from the exterior side of the plunger, to which a depressor 66 is joined.

By inwardly actuating the plunger 60, air is forced from the hollow body handle 22″ and inner tube 14″ into the ducts and veins of the sample collection bag 32, which inflates the bag. The specimen would then be collected as described above. After the sample has been collected, withdrawing the plunger 60 will deflate the bag. Withdrawing the plunger 60 to a position that is further beyond its initial position will cause the bag to cinch closed. Closure will be completed after the plunger 60 has been removed from the hollow body of the handle, and the cinch string is displaced further in the proximal direction, relative to the starting position of the plunger 60.

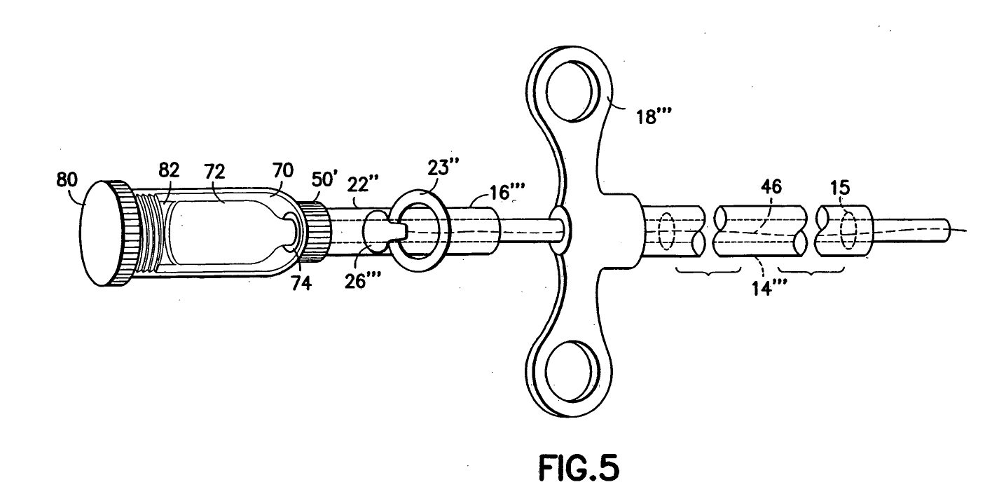

Yet another embodiment is depicted in FIG. 5. In this figure, inner tube 14′″ is provided with a sealing plug 26″ having a pull ring 23″, at a location proximal the outer tube handle 16″. A chamber 70 is sealingly engaged to the open end of the inner tube 14′″ by fitting 50′. The chamber 70 houses a canister 72 of pressurized air, which may, of course, be sterile. A seat 74 is positioned in the chamber adjacent the fitting 50′. The seal (discussed below) may, in one embodiment, be provided with a pressure release valve, to regulate the flow of air into the bag.

A piercing mechanism, such as a tack or blade (not shown), is positioned adjacent the opening of the canister, which is sealed with a frangible diaphragm or other structure that would be opened when sufficient force from the piercing mechanism is applied. Biasing means, such as a spring, can be inserted into the seal so that the frangible diaphragm can be biased away from the piercing mechanism. A threaded screw top 80, provided with a contoured canister holder 82, can be positioned at the opposite end of the canister. The screw top 80 may maintain the canister 72 in place, above the piercing mechanism.

After the inner lube 14′″ has been glidingly displaced towards the distal end of the instrument, which forces the specimen collection bag 32 out of the outer tube, the device is actuated by tightening the screw top, which moves the canister holder 82 down into the canister, forcing the frangible diaphragm into engagement with the piecing mechanism, which breaks the diaphragm, releasing the air from the canister. The air flows through the instrument, and inflates the ducts and veins of the bag in order to open the bag. The sample is then collected. After collection, the bag can be deflated by pulling the sealing plug 26″. Further pulling on the plug may cinch the bag closed.

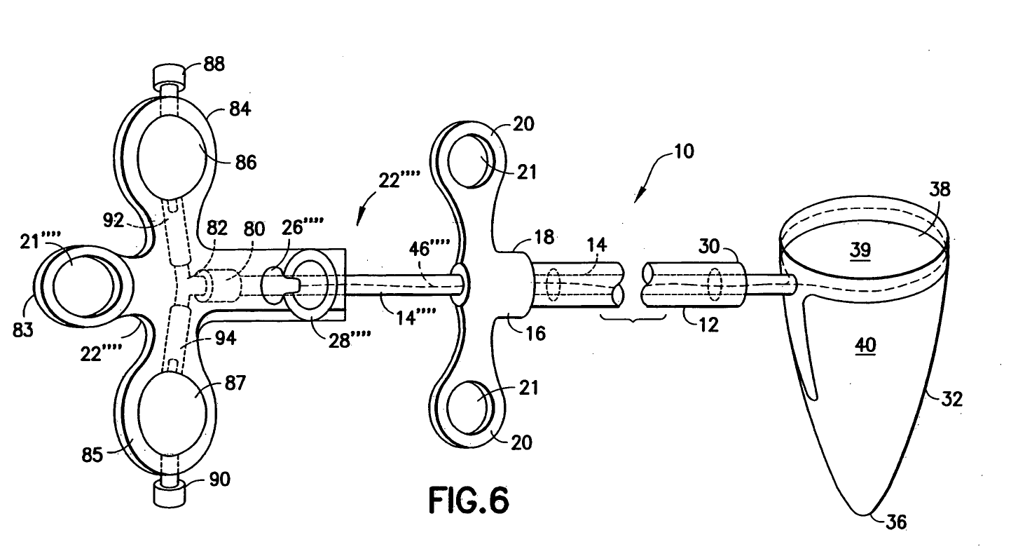

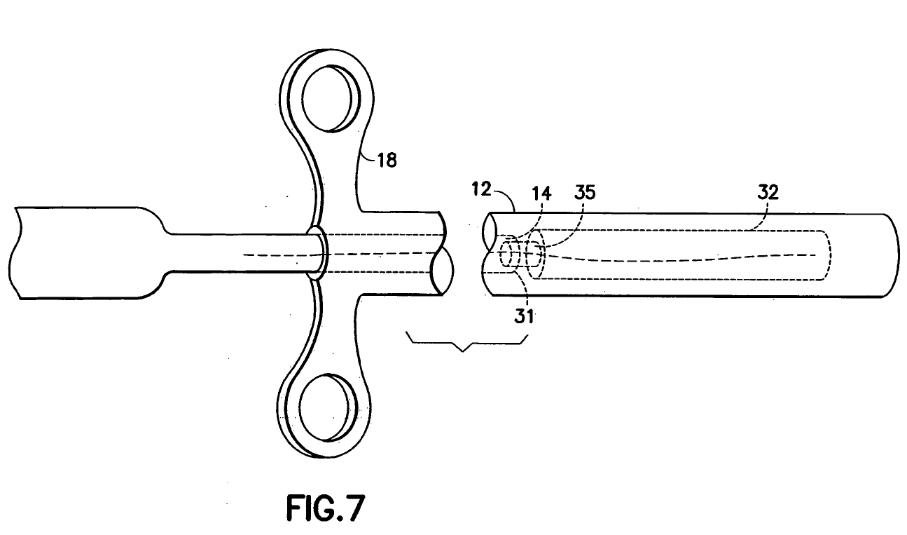

FIG. 6 shows yet another embodiment of the present invention. In this embodiment, inner tube 14″″ is fitted within the proximal end of the handle 22″″. A connector tube 80 sealingly engages the inner tube 14″″ to a three way fitting 82. Handle 22″ is provided with a sealing plug 26″″ having a pull ring 28″″ which seals the inner tube from the outside environment. Cinch string 46″″ is attached to the inner tube facing side of the sealing plug 26″″, and extends through the inner tube 14″″ and stem and around the periphery of the bag, as described above.

Handle 22″″ is provided with three lobes 83, 84, and 85. Lobe 83 is provided with an opening 21″″ through which a finger can be inserted. Lobes 84 and 85 include bulbs 86 and 87. Bulb 86, when actuated, draws air from the environment and inflates the bag. Bulb 87, when actuated, draws air from the bag and deflates the bag

Bulbs 86 and 87 are each provided with reversible valves 88 and 90. Valve 88 is configured so that when opened, compression of bulb 86 causes air to flow through the inner tube and stem, into the ducts (and veins) of the bag. Valve 90 is configured so that when opened, compression of bulb 87 causes air to flow back through the ducts (and veins of the bag, the stem and inner tube) and out through the bulb and valve, in order to deflate the bag. These valves may be screw top valves, which can be opened and closed by rotating them, or they may be provided with plugs, which can be removed or inserted to open or close the valves. Hoses 92, 94 are sealingly engaged with the bulbs 86 and 87 at one end, and to the three-way fitting at another end.

After the inner tube 14″″ has been slidingly displaced towards the distal end of the instrument, which forces the specimen collection bag 32 out of the outer tube 12, the device is actuated by opening the valve 88 on the inflation bulb 86, closing the valve 90 on the deflation bulb 87, and pumping the inflation bulb 86. The air flows through the instrument, and inflates the ducts and veins of the bag in order to open the bag. The sample is then collected. After collection, the bag can be deflated by reversing valves 88 and 90, that is, by closing valve 88 and opening the valve 90, and then pumping the deflation bulb order to remove air from the ducts in the bag. After deflation, the sealing plug 26″″ is pulled from the handle 22″. As the sealing plug 26″″ is tugged, the cinch string 46″ nooses within the top of the sample collection bag 32, which closes the bag. The bag can then be removed from the device by cutting it.

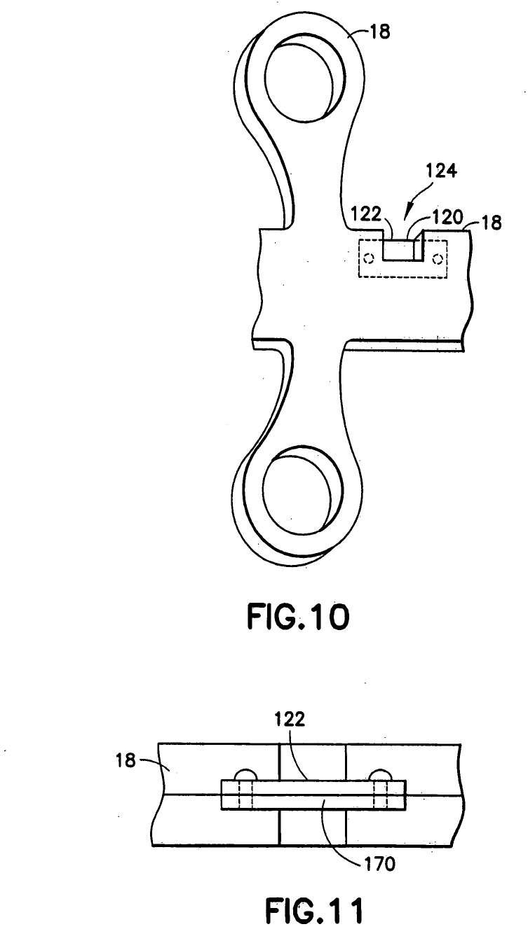

A cutting implement, such as a knife, can be incorporated into these embodiments (e.g., in order to cut the cinch string). Such an arrangement is shown in FIGS. 10 and 11, where knife blade 120, having relatively sharp edge 122, facing towards opening 124, is mounted to the interior side of outer tube 12, in the vicinity of handle 18. The knife may be used to cut the cinch string 46, after the bag has been closed, by frictionally engaging the string with the knife blade for a sufficient period of time.

In constructing the present invention, the stem which joins the bag to the inner tube, and the junction between the stem and bag, may be reinforced with a relatively stronger material, to strengthen this area so that the integrity of the bag is not compromised during any one of deployment, inflation, manipulation and sample collection, cinching or pouch removal. This area may also accommodate graspers or similar instruments. This reinforced material may also supply the structure which attaches and secures the stem and/or the cinch string.

In another embodiment an inflation assisted (inflatable) specimen removal pouch and applicator may include:

Pouch—a pouch, with one or more inflatable chambers, manufactured from a flexible membrane. The pouch will be inflated after deployment. The chambers, when inflated, will open the mouth of the pouch and assist in unfurling the pouch. The pouch may also be deflated by positively removing the air from the chamber. The chamber will also house a cinch string. A heavier material may reinforce the stem area of the chamber. This will strengthen this area so that the integrity of the pouch is not compromised when: deploying, inflating, manipulating, cinching or removing the pouch. This area may also accommodate graspers or similar instruments. This reinforced material may also be a means of attaching and securing the stem and/or the cinch string. The pouch will be removable from the applicator.

Stem—A stem with a base sufficient to support manipulation of the pouch with the detachable tube. The base of the stem will also act as a stop against the outer tube (after deployment) which will assist in removing the detachable tube. The stem may have a seat for a spherical feature attached to the cinch string. Air will pass through stem during inflation and deflation. The cinch string will pass through the stem. The stem may be attached to (or part of) the inner tube as opposed to the pouch.

Cinch String—A cinch string will run circumferentially inside of the upper pouch chamber, out through the stem, up the detachable tube and terminate either on a ring, plug or plunger. The cinch string may or may not be permanently attached to the pouch. If not, a noose type system may be used. Pulling the cinch string will close the pouch. If not permanently attached, the stem will act as a stop. There may be a spherical feature permanently attached to the string. This sphere would enter the stem area upon cinching. This sphere would seat inside the stem therefore sealing the chamber and eliminating or reducing the possibility of air reentering the chamber. This will also assist in guiding the pouch through the incision.

Detachable Tube—a detachable tube acting as:

1. A Pusher—the means of deployment from the outer tube

2. A Handle—used to manipulate the pouch within the body cavity

3. An Airduct—a travel way for air during inflation or deflation

4. A Cinch String Passageway—permitting the cinch string to be drawn freely

The detachable tube will allow the pouch to be left in the cavity until the end of the procedure.

Outer Tube—an outer tube which will house the pouch, seals, and detachable tube.

Knife—a permanently attached knife may be incorporated. This may be used for cutting the cinch string.

In another embodiment a device may be used for removing body tissue from a body cavity during minimally invasive surgery as follows: The device is introduced into the cavity through a port (typically). The pouch is deployed by pushing the inner tube out of the outer tube. After the pouch is deployed the chambers is/are inflated. Inflation methods may vary as described elsewhere in the present application. The tissue is placed in the pouch. The air is then allowed to escape from the chamber (through the stem and inner tube) or is positively removed (methods may vary as described elsewhere in the present application). The cinch string may now be pulled, closing the mouth of the pouch. The cinch string is cut. The device is removed from the port leaving the string. The port may be removed and then the pouch. If the pouch is to be left in the cavity for removal after the procedure, the string may be pulled into the cavity by another port.

In other embodiments, the following describes different body types and means of inflation:

A. A body containing a bulb type pump (or similar method) attached to the detachable tube. The pump is the means for inflation and would have a valve allowing for incremental inflation.

B. The cinch string may terminate on a plug, which will be removable, by pulling on a ring. Removal of the plug will also be the means of allowing air out of the chamber(s) prior to cinching the pouch.

C. Similar to A, above, without the actual body. The cinch string may terminate after exiting the bulb valve; otherwise, a plug would be used similar to that in A, above. The pouch chamber would deflate when the valve is turned.

D. A body consisting of a syringe type plunger pouch chamber (holding more air). Compressing the plunger into the body forces air into the chamber. This is the means of inflation. The size of the plunger body (attached to the detachable tube) would be larger than the pouch chamber (holding more air).

The cinch string would be attached to the bottom of the plunger. The motion of removing the plunger from the body is the means for positively deflating the pouch chamber. The travel of the plunger when deflating will exceed the travel of the plunger when inflating therefore assisting in the closure of the pouch mouth through air removal.

When the plunger is being removed, and has passed it's original start position, the cinch string will begin to close the pouch. Closure will complete after the plunger has been pulled from the plunger body.

E. A body consisting of a chamber that would house a canister of sterile compressed air. Forcing the end of the canister into a seal and piercing mechanism would activate the canister. The mechanism can either: inflate the pouch chamber by fully discharging or could be regulated using a pressure release valve. The cinch string may terminate on a plug with an attached ring. When pulled, this plug would allow the pouch chamber to deflate prior to removal.

F. A body consisting of (2) bulb type pumps (or similar methods). One bulb would inflate the pouch chamber, the other would deflate the chamber by positively removing the air creating a partial vacuum. Upon completion of air removal, the pouch would be cinched. Each pump may have a shutoff for stopping airflow when using the opposing pump. Each pump may connect to the detachable tube via a “Y” shaped connector/fitting. These components may be tied together using (3) connector tubes.

In yet another embodiment, the bag is opened by providing a relatively stiff filament, fiber or wire (hereinafter “relatively stiff fiber”) that runs through the housing and into the duct provided at the top of the bag. The relatively stiff fiber may extend around the duct, returning to the point where the relatively stiff fiber first enters the duct, and may be tied in a noose or knot. In one specific embodiment, the relatively stiff fiber passes through a ring, with the terminal end of the relatively stiff fiber being tied to an end of the ring. The relatively stiff fiber may instead be permanently affixed to the bag or bag seal area, which would eliminate the need for any type of knot.

The relatively stiff fiber exhibits flexibility, though to a limited degree, that permits the relatively stiff fiber to be folded or otherwise compacted to permit the bag to be stored in the end of the outer tube prior to deployment. The bag is deployed from the outer tube by sliding the inner tube towards the distal end of the device, which forces the bag out of the housing. Once the bag is deployed outside of the housing, the stiffness of the relatively stiff fiber that is positioned within the duct of the bag causes the relatively stiff fiber to fully open, thereby expanding the bag, causing it to also open. In one specific embodiment, the relatively stiff fiber is a monofilament, of a material and size sufficient to impart the properties described herein

In yet another embodiment, the duct is provided with a discontinuous channel-like structure in which a number of channel structures are affixed to the top of the bag, through which the relatively stiff fiber is threaded. In yet another specific embodiment, the relatively stiff fiber is stitched through the duct.

In another embodiment the specimen collection instrument may include an outer tube housing an inner tube. Inner tube is provided with an activation knob at a proximal end of the inner tube. The activation knob is sealed by an outer seal such as a plug seal or the like. Relatively stiff fiber passes through the outer seal and at its proximal termination is affixed to the pull knob. The inner tube is positioned within the outer tube. The outer tube defines an open space through its interior, in which inner tube is housed. The outer tube provides a passageway for inner tube to move through. A stop is provided on the outside of the outer tube. O-rings or other sealing mechanism(s) are positioned in the outer tube, between the outer tube and the inner tube in order to create an interference fit between the two. The O-rings seal the space between inner tube and outer tube.

The unactuated sample collection bag resides within the outer tube, near the distal end thereof. The stem extends between the bag and the inner tube and is detachable from at least one of the bag and inner tube. The stem may be formed of a durable, resilient material to support manipulation of the pouch before, during and after sample collection, through manipulation of the inner tube. O-rings positioned within the stem create a seal around the relatively stiff fiber.

The sample collection bag provided with a closed lower end and an open upper end. The collection bag has an inner wall and an outer wall. At least one duct is formed between the inner wall and outer wall. The duct extends around the perimeter of the bag. Relatively stiff fiber, which as indicated above extends through the sealing plug located at the proximal end and is attached to the pull knob, extends in the distal direction through the inner tube, through the stem into the duct of the bag. A ring or washer is positioned in the duct. Relatively stiff fiber passes through the opening in the washer, around the duct, as described above, and the end of relatively stiff fiber is then affixed to the washer. With this arrangement, relatively stiff fiber will tighten around the periphery of the bag in order to close it, when the plug is pulled and the bag is cinched shut. In another embodiment, a noose can be formed, as described above. The noose, or knot, should be sized so that the knot is not drawn up into the stem during the cinching operation. At the appropriate time, when the plug is pulled, the relatively stiff fiber is tightened, cinching the sample collection bag closed.

In operation, the surgical device is introduced into a body cavity (e.g., through a port or opening which has been opened by the surgeon) in a state in which the bag has not been actuated. The pouch is deployed by pushing the knob to slide the inner tube through the outer tube, causing the bag to be pushed out of the outer tube. When the bag passes out of the distal end of the outer tube, the relatively stiff fiber flexes outwardly to open the upper end of the bag. This opens the bag to a state in which it is sufficiently open to receive a sample of tissue or other specimen.

In alternative arrangements for passing the relatively stiff fiber through the duct, the duct may be constructed of a number of discontinuous channels and/or the relatively stiff fiber may be woven through the duct and/or upper end of the bag.

In another embodiment the relatively stiff fiber may be disposed on/within the specimen retrieval bag to aid in unfurling the specimen retrieval bag.

In another embodiment the cinch string and/or relatively stiff fiber may include or be made entirely of an elastic member (e.g., an elastic band).

In another embodiment the invention may be made smaller than conventional devices are typically made. For example, while a conventional device may typically be made to be inserted through a port or trocar of approximately 10 mm, the present invention may be made to be inserted through a port or trocar of approximately 5 mm.

While particular embodiments of the present invention have been shown and described herein, it should be clear to those skilled in the art that such embodiments are provided by way of example only. Numerous variations, changes, and substitutions will now occur to those skilled in the art without departing from the invention. For example, the invention may be utilized in any desired minimally invasive surgery or in any desired open surgery. Further, a relatively stiff fiber may be used in conjunction with gas inflation (e.g., where gas pressure alone is not enough to inflate, unfurl, and/or support the specimen collection bag). Further still, the specimen collection bag may have one or more side or bottom openings (e.g., for use in conjunction with or in place of one or more top openings). Further still, the specimen collection bag may take any desired shape (e.g., tapered, conical, square, rectangular). Further still, the invention may be used without a cinch string.

What is claimed is:

1. A specimen collection apparatus, comprising:
a collapsible specimen retrieval bag having an open end which can be cinched shut to contain a specimen therein;
a housing for holding the specimen retrieval bag in a collapsed state;
a mechanism for deploying the specimen retrieval bag from the housing; and
a mechanism for shaping the open end of the specimen retrieval bag, after the specimen retrieval bag is deployed from the housing, to form an aperture large enough to receive the specimen into the specimen retrieval bag;
wherein the mechanism for shaping the open end of the specimen retrieval bag includes at least one substantially gas-tight duct associated with the specimen retrieval bag, which duct is disposed at a position selected from the group of: (a) on an inside surface of the specimen retrieval bag; (b) on an outside surface of the specimen retrieval bag; and (c) between the inside surface of the specimen retrieval bag and the outside surface of the specimen retrieval bag.

2. The apparatus of claim 1, wherein the duct is disposed adjacent the open end of the specimen retrieval bag.

3. The apparatus of claim 2, wherein the duct forms a generally circular ring when gas pressure in the duct is greater than gas pressure outside the duct.

4. The apparatus of claim 3, further comprising a mechanism for supplying gas to the duct in order to inflate the duct.

5. The apparatus of claim 4, wherein the mechanism for supplying the gas to the duct is adapted to provide incremental inflation of the duct.

6. The apparatus of claim 5, wherein the mechanism for supplying the gas to the duct is selected from the group of: (a) an electrical pump; (b) a hand-operated pump; (c) a container of compressed gas; and (d) a source within an operating room.

7. The apparatus of claim 6, wherein the hand operated pump is selected from the group of: (a) a bulb-type pump; and (b) a syringe.

8. The apparatus of claim 3, further comprising a mechanism for permitting gas pressure in the duct to be reduced in order to deflate the duct.

9. The apparatus of claim 8, wherein the mechanism for permitting gas pressure in the duct to be reduced is adapted to provide incremental deflation of the duct.

10. The apparatus of claim 9, wherein the mechanism for permitting gas pressure in the duct to be reduced comprises a valve.

11. The apparatus of claim 3, further comprising a mechanism for deflating the duct by decreasing gas pressure therein.

12. The apparatus of claim 11, wherein the mechanism for deflating the duct comprises a mechanism for pumping gas out of the duct.

13. The apparatus of claim 12, wherein the mechanism for pumping gas out of the duct is adapted to provide incremental deflation of the duct.

14. The apparatus of claim 13, wherein the mechanism for pumping gas out of the duct is selected from the group of: (a) an electrical pump; (b) a hand-operated pump; and (c) a source within an operating room.

15. The apparatus of claim 14, wherein the hand-operated pump is selected from the group of: (a) a bulb-type pump; and (b) a syringe.

16. The apparatus of claim 1, wherein the gas is air.

17. The apparatus of claim 1, further comprising a mechanism for cinching shut the open end of the specimen retrieval bag.

18. The apparatus of claim 17, wherein the mechanism for cinching shut the open end of the specimen retrieval bag includes a string running at least partially through the duct, which string may be pulled to cinch shut the open end of the specimen retrieval bag.

19. The apparatus of claim 1, wherein the housing comprises a first elongated tube with two ends.

20. The apparatus of claim 19, wherein the mechanism for deploying the specimen retrieval bag comprises a second elongated tube with an outside diameter smaller than an inside diameter of the first elongated tube, wherein the second elongated tube is configured to push the specimen retrieval bag out of one of the ends of the first elongated tube.

21. The apparatus of claim 1, further comprising a knife disposed on the housing.

22. The apparatus of claim 21, wherein the knife is disposed on an end of the housing opposite that from which the specimen retrieval bag is deployed.

23. A specimen collection apparatus, comprising:
a collapsible specimen retrieval bag having an open end which can be cinched shut to contain a specimen therein;
a housing for holding the specimen retrieval bag in a collapsed state, which housing comprises a first elongated tube with two ends;
a mechanism for deploying the specimen retrieval bag from the housing, which mechanism for deploying the specimen retrieval bag from the housing comprises a second elongated tube with an outside diameter smaller than an inside diameter of the first elongated tube, wherein the second elongated tube is configured to push the specimen retrieval bag out of one of the ends of the first elongated tube;
a substantially gas-tight duct associated with the specimen retrieval bag for shaping the open end of the specimen retrieval bag, after the specimen retrieval bag is deployed from the housing, to form an aperture large enough to receive the specimen into the specimen retrieval bag;
a mechanism for inflating the duct by supplying gas to the duct;
a mechanism for deflating the duct by permitting gas pressure in the duct to be reduced; and
a mechanism for cinching shut the open end of the specimen retrieval bag;
wherein the duct is disposed at a position selected from the group of: (a) on an inside surface of the specimen retrieval bag; (b) an outside surface of the specimen retrieval bag; and (c) between the inside surface of the specimen retrieval bag and the outside surface of the specimen retrieval bag;
wherein the duct is disposed adjacent the open end of the specimen retrieval bag; and
wherein the duct forms a generally circular ring when gas pressure in the duct is greater than gas pressure outside the duct.

24. The apparatus of claim 23, wherein the mechanism for cinching shut the open end of the specimen retrieval bag includes a string running at least partially through the duct, which string may be pulled to cinch shut the open end of the specimen retrieval bag.

25. The apparatus of claim 24, wherein: (a) the string runs through the first elongated tube and the second elongated tube; and (b) gas is supplied to the duct through the first elongated tube and the second elongated tube.

26. The apparatus of claim 25, further comprising a mechanism for pumping gas out of the duct.

27. The apparatus of claim 26, wherein gas is pumped out of the duct through the first elongated tube and the second elongated tube.

28. The apparatus of claim 24, further comprising a stem in fluid communication with the duct, wherein gas passes through the stem during inflation and deflation.

29. The apparatus of claim 28, wherein the string runs through the stem.

30. The apparatus of claim 29, wherein the stem is attached to the specimen retrieval bag at a reinforced portion of the specimen retrieval bag.

31. The apparatus of claim 24, wherein the mechanism for deflating the duct by permitting gas pressure in the duct to be reduced comprises a valve having a gas blocking element seatable in an interference fit with a seating surface.

32. The apparatus of claim 31, wherein the valve is operatively connected to the string such that the valve permits gas pressure in the duct to be reduced after the string is pulled.

33. The apparatus of claim 24, further comprising a backflow prevention mechanism operatively connected to the string for substantially prohibiting a flow of gas back into the duct after the string is pulled past a predetermined position during the cinching shut of the open end of the specimen retrieval bag.

34. The apparatus of claim 33, wherein the backflow prevention mechanism comprises a valve having a gas blocking element seatable in an interference fit with a seating surface.

35. The apparatus of claim 23, further comprising a mechanism for unfurling the specimen retrieval bag, after the specimen retrieval bag is deployed from the housing.

36. The apparatus of claim 35, wherein the mechanism for unfurling the specimen retrieval bag comprises at least one vein extending from the duct, which vein is in fluid communication with the duct.

37. The apparatus of claim 36, wherein the vein extends generally away from the open end of the specimen retrieval bag and towards a closed end of the specimen retrieval bag.
